(19) United States
(12) Patent Application Publication
(10) Pub. No.: US 2014/0228797 A1
(43) Pub. Date: Aug. 14, 2014

(54) Diaper's Pull-N-Pouch

(71) Applicants: Tahisha Beresford, Orlando, FL (US); Gary Wheeler, Orlando, FL (US)

(72) Inventors: Tahisha Beresford, Orlando, FL (US); Gary Wheeler, Orlando, FL (US)

(21) Appl. No.: 14/169,461

(22) Filed: Jan. 31, 2014

Related U.S. Application Data
(60) Provisional application No. 61/764,004, filed on Feb. 13, 2013.

(51) Int. Cl. A61F 13/49; A61F 13/84

(52) U.S. Cl. CPC A61F 13/49; A61F 13/84
USPC 604/385.06

(57) ABSTRACT

A baby wipe device is disclosed that attaches baby wipes to individual diapers, holding them in an easily accessible pouch. The baby wipe device comprises a diaper component, a pocket component secured to the diaper component, and a plurality of wipe components positioned within the pocket component. The pocket component comprises a hollow interior and an opening formed therein, which defines an access to and from the hollow interior. The plurality of wipe components are positioned within the hollow interior for easy access by the user. The plurality of wipe components are formed of a liquid absorbent material and are sufficiently soft in texture to be applied to the skin of an infant or child. Typically, the pocket component comprises a pull-back seal which covers the opening. A user would pull-back the seal and reach into the opening to remove wipe components as needed.

### CROSS-REFERENCE

This application claims priority from Provisional Patent Application Ser. No. 61/764,004 filed Feb. 13, 2013.

### BACKGROUND

Changing a baby's diaper may be difficult if parents or caregivers don't have easy access to baby wipes. Many people carry baby wipes in their diaper bag to avoid the is problem, but they can weigh down the bag and can be expensive to buy over time. An effective solution is necessary.

The present invention provides parents and caregivers with a uniquely designed diaper with a pouch on the front to house baby wipes, and allows easy access to baby wipes when changing a diaper or cleaning up a mess. The baby wipe device simplifies the overall diaper-changing process, and eliminates the need to carry an additional container of baby wipes in a diaper bag. The baby wipe device is ideal for babies and infants between ages 0 and 3. Any parent or caregiver of a young child or infant would benefit from the convenience and efficiency this device provides.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a baby wipe device that attaches baby wipes to individual diapers, holding them in an easily accessible pouch, thus allowing easy access to baby wipes when changing a diaper. The baby wipe device comprises a diaper component, at least one pocket component secured to the diaper component, and a plurality of wipe components positioned within the at least one pocket component. The diaper component is any typical prior art diaper as is known in the art. The at least one pocket component comprises a hollow interior for the containment of the wipe components. Further, the pocket component comprises an opening formed therein, which defines an access to and from the hollow interior of the pocket component. The plurality of wipe components are positioned within the hollow interior of the pocket component for easy access by the user. The plurality of wipe components are formed of a liquid absorbent material, such as cotton, etc., and are sufficiently soft in texture to be applied to the skin of an infant or child. Typically, the plurality of wipe components are pre-moistened with a cleaning solution as is known in the art.

In a preferred embodiment, the pocket component comprises a pull-back seal which secures (or covers) the opening. A user would pull-back (or remove) the seal and reach into the opening to remove wipe components from the pocket component as needed. Further, the plurality of wipe components can comprise aloe vera and/or vitamin E, or any other suitable moisturizer or health solution as is known in the art without affecting the overall concept of the invention.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a perspective view of the baby wipe device in accordance with the disclosed architecture.

FIG. 2 illustrates a perspective view of the baby wipe device with the pocket component being opened in accordance with the disclosed architecture.

FIG. 3 illustrates a perspective view of the baby wipe device with a wipe component in the pocket component in accordance with the disclosed architecture.

FIG. 4 illustrates a perspective view of the baby wipe device with the wipe component being removed from the pocket component in accordance with the disclosed architecture.

FIG. 5 illustrates a perspective view of the baby wipe device in use in accordance with the disclosed architecture.

### DESCRIPTION OF PREFERRED EMBODIMENTS

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention provides parents and caregivers with a uniquely designed diaper with a pouch on the front to house baby wipes, and allows easy access to baby wipes when changing a diaper or cleaning up a mess. The baby wipe device simplifies the overall diaper-changing process, and eliminates the need to carry an additional container of baby wipes in a diaper bag. The baby wipe device is ideal for babies and infants between ages 0 and 3.

The baby wipe device comprises a diaper component, a pocket component secured to the diaper component, and a plurality of wipe components positioned within the pocket component. The pocket component comprises a hollow interior and an opening formed therein, which defines an access to and from the hollow interior. The plurality of wipe components are positioned within the hollow interior for easy access by the user. The plurality of wipe components are formed of a liquid absorbent material and are sufficiently soft in texture to be applied to the skin of an infant or child. Typically, the pocket component comprises a pull-back seal which covers the opening. A user would pull-back the seal and reach into the opening to remove wipe components as needed.

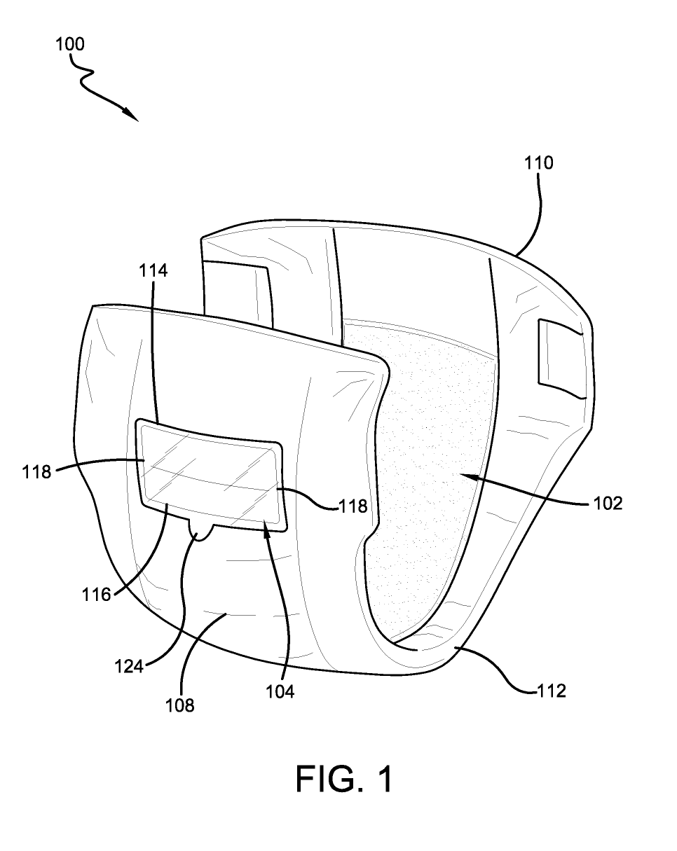

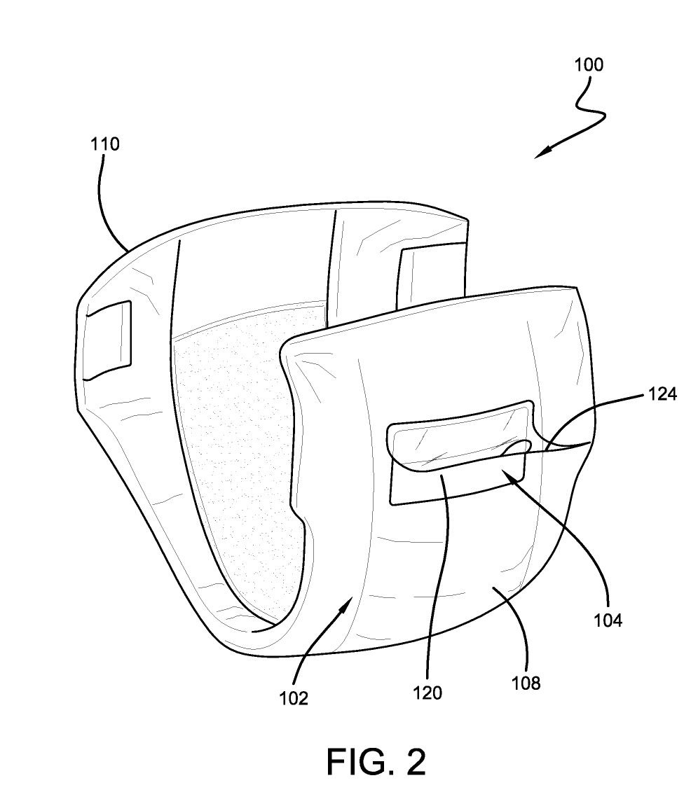

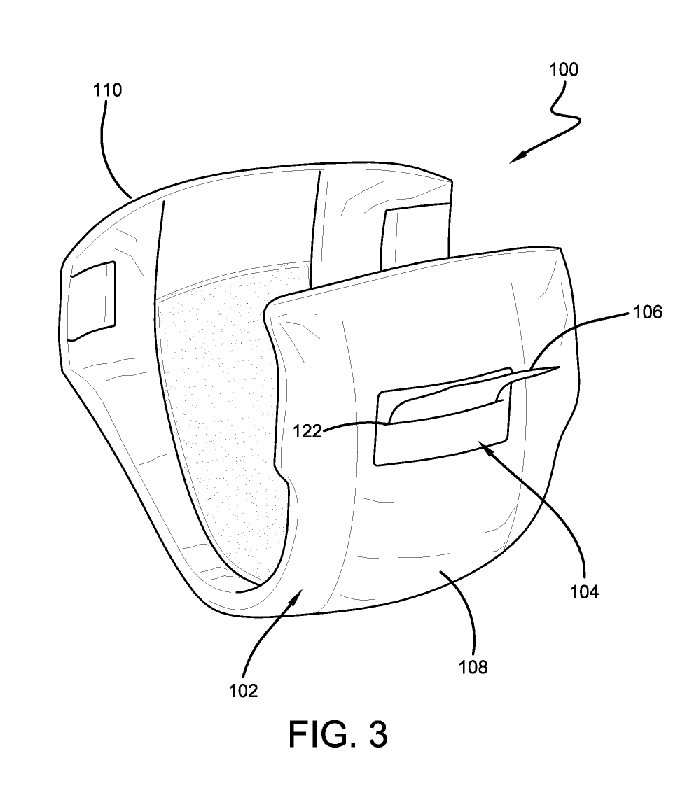

Referring initially to the drawings, FIGS. 1-3 illustrate the baby wipe device 100 that attaches baby wipes to individual diapers, holding them in an easily accessible pouch, thus allowing easy access to baby wipes when changing a diaper. The baby wipe device 100 comprises a diaper component 102, at least one pocket component 104 secured to the diaper component 102, and a plurality of wipe components 106 positioned within the at least one pocket component 104 (as shown in FIG. 1).

The diaper component 102 is any typical prior art diaper as is known in the art, such as disposable diapers, cloth diapers, infant diapers, toddler diapers, disposable training pants, adult diapers, etc. The diapers can be releasably secured on opposing sides to permit access, or can be pull-up diapers or training pants with secure sides. The diaper component 102 comprises a front end 108, a back end 110, and opposing sides 112, and would generally be constructed of cotton fiber, polyester, rayon, etc., though any other suitable material may be used to manufacture the diaper component 102 as is known in the art without affecting the overall concept of the invention.

The at least one pocket component 104 comprises a first end 114, a second end 116, opposing sides 118, and a hollow interior 120 for the containment of the wipe components 106. Typically, the pocket component 104 can be any suitable shape as is known in the art without affecting the overall concept of the invention, such as rectangular, square, circular, oval, triangular, etc. The pocket component 104 would generally be constructed of recycled plastics, or homogeneous plastics, such as polyvinyl chloride (PVC), acrylonitrile butadiene styrene (ABS), polycarbonate (PC), composite polymers, etc., though any other suitable material may be used to manufacture the pocket component 104 as is known in the art without affecting the overall concept of the invention.

The pocket component 104 can also comprise a variety of colors and designs to suit user and manufacturing preference.

Any number of pocket components 104 can be secured to the diaper component 102, depending on the needs and wants of a user. Typically, only one pocket component 104 is secured to the diaper component 102. Further, the pocket component 104 is generally secured to the front end 108 of the diaper component 102, but can be secured to any suitable place on the diaper component 102, depending on the needs and wants of a user. The pocket component 104 is typically secured via gluing to the diaper component 102, but can be secured via any other suitable securing means as is known in the art, such as fasteners, welding, etc. The pocket component 104 could also be manufactured as an integral piece to the diaper component 102, without affecting the overall concept of the invention.

Furthermore, the pocket component 104 comprises an opening 122 formed therein, which defines an access to and from the hollow interior 120 of the pocket component 104. The plurality of wipe components 106 are positioned within the hollow interior 120 of the pocket component 104 for easy access by the user. Further, the pocket component 104 comprises a pull-back seal 124 which secures (or covers) the opening 122. Any suitable removably attached seal can be used to secure the opening 122 as is known in the art without affecting the overall concept of the invention. A user would pull-back (or remove) the seal 124 and reach into the opening 122 to remove wipe components 106 from the pocket component 104 as needed.

A plurality of wipe components 106 are positioned within the hollow interior 120 of the pocket component 104. Typically, up to five wipe components 106 are contained within the hollow interior 120 of the pocket component 104, however any suitable number of wipe components 106 can be contained within the pocket component 104 as is known in the art without affecting the overall concept of the invention. The plurality of wipe components 106 are formed of a liquid absorbent material, such as cotton, etc., and are sufficiently soft in texture to be applied to the skin of an infant or child. Typically, the plurality of wipe components 106 are pre-moistened with a cleaning solution as is known in the art. Further, the plurality of wipe components 106 can comprise aloe vera and/or vitamin E, or any other suitable moisturizer or health solution as is known in the art without affecting the overall concept of the invention.

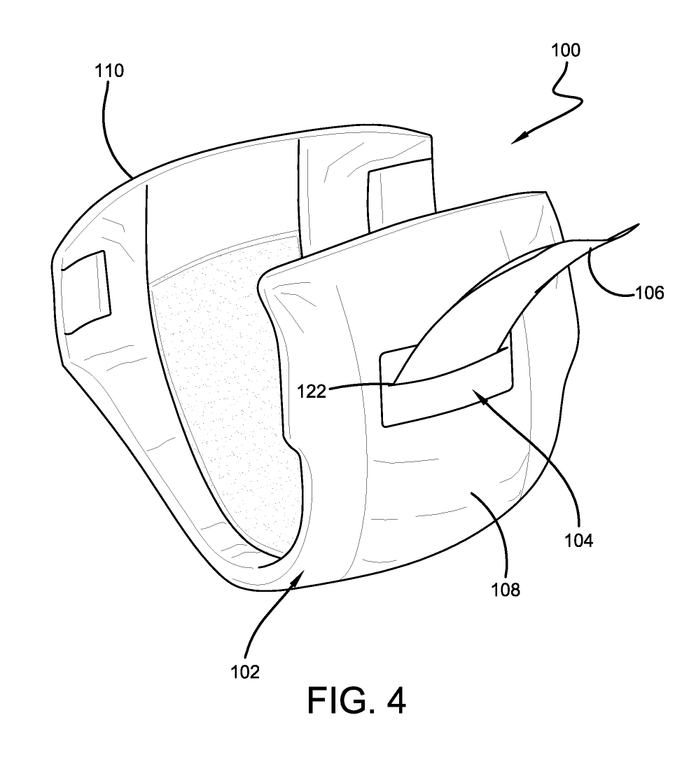

FIG. 4 illustrates the baby wipe device 100 with the wipe component 106 being removed from the pocket component 104. As stated supra, a plurality of wipe components 106 are positioned within the hollow interior 120 of the pocket component 104 (i.e., typically up to five wipe components 106 are contained within the hollow interior 120). The plurality of wipe components 106 are formed of a liquid absorbent material, such as cotton, etc., and are sufficiently soft in texture to be applied to the skin of an infant or child. Typically, the plurality of wipe components 106 are pre-moistened with a cleaning solution, and can also comprise aloe vera, vitamin E, or other suitable moisturizers/health solutions as is known in the art.

The plurality of wipe components 106 are positioned within the hollow interior 120 of the pocket component 104 for easy access by the user. Further, the pull-back seal 124 secures (or covers) the opening 122. A user would pull-back (or remove) the seal 124 and reach into the opening 122 to remove wipe components 106 from the pocket component 104 as needed.

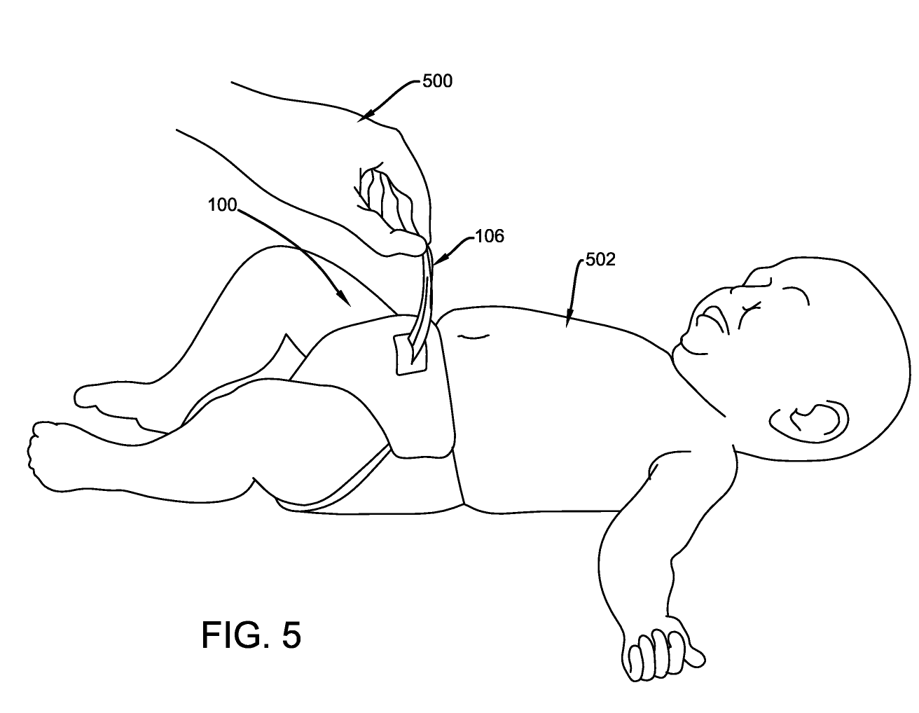

FIG. 5 illustrates the baby wipe device 100 in use. As stated supra, the baby wipe device 100 comprises a diaper component 102, a pocket component 104 secured to the diaper component 102, and a plurality of wipe components 106 positioned within the pocket component 104. The pocket component 104 comprises a hollow interior 120 and an opening 122 formed therein, which defines an access to and from the hollow interior 120.

The plurality of wipe components 106 are positioned within the hollow interior 120 for easy access by the user 500. The plurality of wipe components 106 are formed of a liquid absorbent material and are sufficiently soft in texture to be applied to the skin of an infant or child 502. Typically, the pocket component 104 comprises a pull-back seal 124 which covers the opening 122. A user 500 would pull-back the seal 124 and reach into the opening 122 to remove wipe components 106 as needed.

In operation, a user 500 would choose the size and/or specific design of the baby wipe device 100 that meets their needs and/or wants, depending on the age and size of the infant or child which will wear the baby wipe device 100. The user 500 would then secure the pocket component 104 to the front end 108 of the diaper component 102. The user would then secure the diaper component 102 to the infant or child 502, as a typically diaper is usually placed on an infant or child.

Once the infant or child 502 soils the diaper component 102, the user 500 pulls-back the seal 124 from the pocket component 104, exposing the opening 122. The user 500 can then reach in the opening 122 and remove a desired number of wipe components 106, depending on the user's needs and/or desires. The user 500 then removes the diaper component 102 from the infant or child 502 and cleans the infant or child 502 with wipe components 106. Once clean, the user 500 replaces the soiled diaper component 102 with a new (and clean) diaper component 102 and dresses the infant or child 502 as usual. Thus, the device 100 provides parents and caregivers with a uniquely designed diaper with a pouch on the front to house baby wipes, and allows easy access to baby wipes when changing a diaper or cleaning up a mess.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A baby wipe device for use with a diaper, comprising:
a diaper component comprising a front end, a back end, and opposing sides;
at least one pocket component secured to the front end of the diaper component comprising a hollow interior; and
a plurality of wipe components positioned within the hollow interior of the at least one pocket component.

2. The baby wipe device of claim 1, wherein the diaper component is a disposable infant diaper.

3. The baby wipe device of claim 2, wherein the at least one pocket component is secured to the front end of the diaper component via gluing.

4. The baby wipe device of claim 1, wherein the at least one pocket comprises an opening formed therein, defining an access to and from the hollow interior of the at least one pocket.

5. The baby wipe device of claim 4, wherein the at least one pocket comprises a pull-back seal which covers the opening.

6. The baby wipe device of claim 1, wherein the at least one pocket is made of recycled plastic.

7. The baby wipe device of claim 1, wherein the plurality of wipe components are formed of a liquid absorbent material and are sufficiently soft in texture to be applied to the skin of an infant or child.

8. The baby wipe device of claim 7, wherein the plurality of wipe components are pre-moistened with a cleaning solution.

9. The baby wipe device of claim 1, wherein the plurality of wipe components comprise aloe vera.

10. The baby wipe device of claim 7, wherein the plurality of wipe components comprise vitamin E.

11. The baby wipe device of claim 1, wherein the at least one pocket holds up to five wipe components.

12. A baby wipe device for use with a disposable infant diaper, comprising:
a disposable diaper component comprising a front end, a back end, and opposing sides;
at least one pocket component secured to the front end of the disposable diaper component comprising a hollow interior and an opening formed therein defining an access to and from the hollow interior; and
a plurality of wipe components positioned within the hollow interior of the at least one pocket component.

13. The baby wipe device of claim 12, wherein the at least one pocket comprises a pull-back seal which covers the opening.

14. The baby wipe device of claim 12, wherein the at least one pocket component is secured to the front end of the diaper component via gluing.

15. The baby wipe device of claim 12, wherein the plurality of wipe components are formed of a liquid absorbent material and are sufficiently soft in texture to be applied to the skin of an infant or child.

16. The baby wipe device of claim 15, wherein the plurality of wipe components are pre-moistened with a cleaning solution.

17. The baby wipe device of claim 16, wherein the plurality of wipe components comprise aloe vera and Vitamin E.

18. A baby wipe device for use with a disposable infant diaper, comprising:
a disposable diaper component comprising a front end, a back end, and opposing sides;
at least one pocket component secured via gluing to the front end of the disposable diaper component comprising a hollow interior and an opening formed therein defining an access to and from the hollow interior;
a plurality of wipe components positioned within the hollow interior of the at least one pocket component, wherein the plurality of wipe components are formed of a liquid absorbent material and are sufficiently soft in texture to be applied to the skin of an infant or child; and
a pull-back seal which covers the opening of the at least one pocket component.

19. The baby wipe device of claim 18, wherein the at least one pocket is made of recycled plastic.

20. The baby wipe device of claim 18, wherein the plurality of wipe components are pre-moistened with at least one of a cleaning solution, aloe vera, and vitamin E.
